(19) United States
(12) Patent Application Publication
(10) Pub. No.: US 2022/0387359 A1
(43) Pub. Date: Dec. 8, 2022

(54) SYNERGISTIC INHIBITORS OF CREB-MEDIATED GENE TRANSCRIPTION

(71) Applicant: OREGON HEALTH & SCIENCE UNIVERSITY, Portland, OR (US)

(72) Inventors: Xiangshu Xiao, Portland, OR (US); Funchun Xie, Portland, OR (US); Bingbing Li, Portland, OR (US)

(21) Appl. No.: 17/771,730

(22) PCT Filed: Oct. 23, 2020
(86) PCT No.: PCT/US2020/057218

§ 371 Date: Apr. 25, 2022

Related U.S. Application Data
(60) Provisional application No. 62/925,582, filed on Oct. 24, 2019.

(51) Int. Cl. A61K 31/167; A61P 35/00; A61K 45/06

(52) U.S. Cl. CPC A61K 31/167; A61P 35/00; A61K 45/06

(57) ABSTRACT

Disclosed herein are uses for a compound of Formula (I), below, or a pharmaceutically acceptable salt thereof:

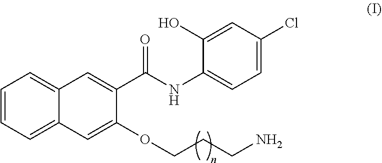

wherein n is an integer selected from the group of 0, 1, 2, 3, and 4, or a pharmaceutically acceptable salt thereof, for enhancing the effect of an anti-cancer agent, such as an inhibitor of CREB-mediated gene transcription.

### RELATED APPLICATIONS

This application claims priority to U.S. provisional patent application No. 62/925,582, filed Oct. 24, 2019.

### ACKNOWLEDGEMENT OF GOVERNMENT SUPPORT

This invention was made with government support under R01 GM122820 awarded by the National Institutes of Health. The government has certain rights in the invention.

### FIELD OF THE INVENTION

The present invention concerns synergistic activity of therapeutic agents associated with the inhibition of CREB-mediated gene transcription.

### BACKGROUND OF THE INVENTION

cAMP-response element binding protein (CREB) is a critical nuclear transcription factor enabling cells to detect and respond to the extracellular microenvironment through transcriptional regulation.¹ In an unstimulated cell, CREB constitutively binds to the cAMP-response elements (CRE) in the genome in a transcriptionally inactive state. Its transcription activity is activated upon phosphorylation mediated by a number of protein serine/threonine kinases, which are collectively called CREB kinases.¹ Once phosphorylated, CREB can bind to the transcription coactivators including CREB-binding protein (CBP) and its paralog p300 through their KIX domain and kinase-inducible domain (KID) in CREB.²⁻³ The known CREB kinases include protein kinase A (PKA), protein kinase B (PKB/Akt), mitogen-activated protein kinases (MAPKs) and ribosomal S6 kinase (pp90^{RSK}). The CREB's transcription activity and phosphorylation status are tightly regulated in normal cells allowing activation of CREB in a timely and pulsative fashion. The key signaling event to turn off CREB's transcription activity is dephosphorylation. Three phosphatases including protein phosphatase 1 (PP1),⁴ protein phosphatase 2 (PP2),⁵ and phosphatase and tensin homolog (PTEN)⁶ are known to dephosphorylate CREB and consequently inactivate CREB's transcription activity.

The CREB kinases are often over-activated in cancer cells through either mutation or excessive growth signals. On the other hand, the phosphatases that can dephosphorylate CREB are known as potent tumor suppressors and often inactivated in the cancer cells through both genetic and non-genetic mechanisms.⁷ As a consequence of the positive and negative regulation of CREB, cancer tissues from different organs have been consistently shown to present higher expression levels of CREB and/or phosphorylated CREB than their normal counterparts.⁸ These cancers include acute myeloid leukemia (AML), breast cancer, prostate cancer, non-small cell lung cancer, glioblastoma (GBM) and kidney cancer.⁸⁻¹⁰ Very interestingly, the CREB signaling has also been shown to mediate melanoma cell resistance to BRAF(V600E) and MAPK inhibitors as well as the progression of castration-resistant prostate cancer.¹¹⁻¹²

Therefore, targeting CREB has been pursued an attractive strategy to develop novel cancer therapies that may ultimately overcome therapy resistance^{8, 13-15}

### SUMMARY OF THE INVENTION

Described are uses for a compound of Formula (I), below, or a pharmaceutically acceptable salt thereof:

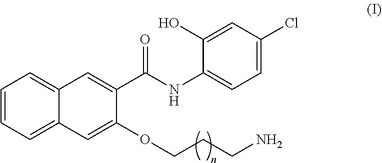

wherein n is an integer selected from the group of 0, 1, 2, 3, and 4, or a pharmaceutically acceptable salt thereof, for enhancing the effect of an anti-cancer agent, such as an inhibitor of CREB-mediated gene transcription.

### BRIEF DESCRIPTION OF THE MANY VIEWS OF THE DRAWINGS

FIG. 1 represents HPLC chromatograms of compound 3 incubated in complete cell culture media for 5 and 30 minutes.

FIG. 2A represents inhibition of CREB-mediated gene transcription by individual compounds.

FIG. 2B represents inhibition of CREB-mediated gene transcription by compound combinations.

FIG. 3 represents synergistic inhibition of CREB-mediated gene transcription in HEK 293T cells by compounds 666-15 and 11 (653-47).

FIG. 4 represents comparative CREB inhibition activity exhibited by Compound 666-15 alone and in combination with Compound 653-47.

FIG. 5 graphs combination indexes (CI) of Compound 666-15 and Compound 11 in inhibiting CREB-mediated gene transcription. The CI values were calculated using Chou-Talalay method from the data in FIG. 2. Fa represents affected fraction.

FIG. 6 represents synergistic anti-proliferative effect of 666-15 and compound 11 in MDA-MB-231 cells. The cells were incubated with the indicated concentrations of 666-15 along with or without 11 (0.5 or 1.0 μM) for 72 h. Then the viable cells were quantified by the MTT reagent. The numbers above each bar indicated the CI values for each combination. The data are presented as mean+SEM. The SEM was from one representative experiment performed in duplicates.

### DETAILED DESCRIPTION OF THE INVENTION

Recently, we described the identification of 666-15 (3-(3-aminopropoxy)-N-(2-((3-((4-chloro-2-hydroxyphenyl)carbamoyl)naphthalen-2-yl)oxy)ethyl)-2-naphthamide) as a potent and selective inhibitor of CREB-mediated gene transcription.^{13, 16} Among a panel of other transcription factors, it was found that 666-15 selectively inhibited CREB-mediated gene transcription.¹⁶ With 666-15 as a chemical tool, we showed that in vivo systemic inhibition of CREB was well-tolerated and produced efficacious anti-breast cancer activity.^{13, 16} Others have also shown that 666-15 inhibited AML, GBM and pancreatic cancer cell growth in different preclinical models.¹⁷⁻¹⁹ Furthermore, recent studies have shown that 666-15 could target the tumor microenvironment to present beneficial anticancer effect. For example, 666-15 was shown to inhibit pro-tumoric IL-6 expression from cancer-associated fibroblasts²⁰ and reverse the immunosuppressive effect of myeloid-derived suppressor cells (MDSC),²¹ suggesting the potential of 666-15 to modulate tumor microenvironment and immunotherapy.

In the chemical structures below for CREB inhibitors 666-15 (3-(3-aminopropoxy)-N-(2-((3-((4-chloro-2-hydroxyphenyl)carbamoyl)naphthalen-2-yl)oxy)ethyl)-2-naphthamide), 1 (3-(3-aminopropoxy)-N-(3-((3-((4-chloro-2-hydroxyphenyl)carbamoyl)naphthalen-2-yl)oxy)propyl)-2-naphthamide), 2 (2-(3-(3-aminopropoxy)-2-naphthamido)-5-chlorophenyl 3-(3-aminopropoxy)-2-naphthoate), and the designed ester prodrug 3 (2-(3-(2-aminoethoxy)-2-naphthamido)-5-chlorophenyl 3-(3-aminopropoxy)-2-naphthoate), the migrating acyl group is highlighted in the lighter gray shade.

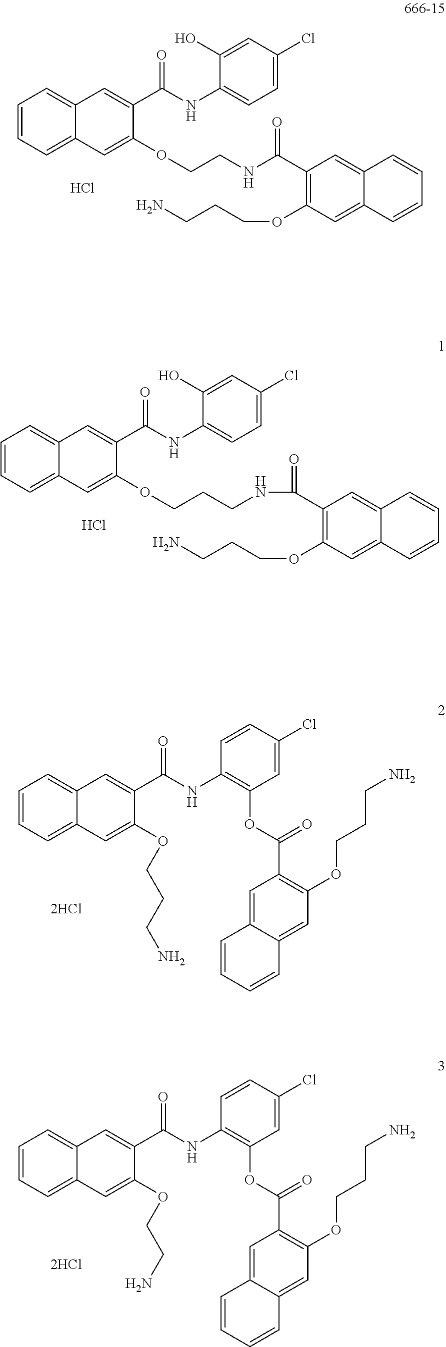 [SMILES: NCCCOc1cc2ccccc2cc1C(=O)NCCOc1cc2ccccc2cc1C(=O)Nc1ccc(Cl)cc1O ; Cl ; NCCCOc1cc2ccccc2cc1C(=O)NCCCOc1cc2ccccc2cc1C(=O)Nc1ccc(Cl)cc1O ; Cl ; NCCCOc1cc2ccccc2cc1C(=O)Nc1ccc(Cl)cc1OC(=O)c1cc2ccccc2cc1OCCCN ; NCCCOc1cc2ccccc2cc1C(=O)Oc1cc(Cl)ccc1NC(=O)c1cc2ccccc2cc1OCCN]

Compound 666-15 was derived from optimization of a less potent derivative 1,¹³ which was initially identified through our discovery of a rapid long-range O,N-acyl transfer reaction of the ester compound 2.¹⁵ One advantage of compound 2 over compound 1 is its high aqueous solubility (>100 mg/mL in ddIH₂O).¹⁵ In this report, we took advantage of the fact of high aqueous solubility of 2 and designed a corresponding ester compound 3 as a potential prodrug of 666-15 with improved aqueous solubility. Unexpectedly, compound 3 was found to undergo a different conversion reaction at physiologically relevant pH (7.40). Furthermore, we discovered a novel compound that can potentiate 666-15's inhibitory effect against CREB-mediated gene transcription in living cells.

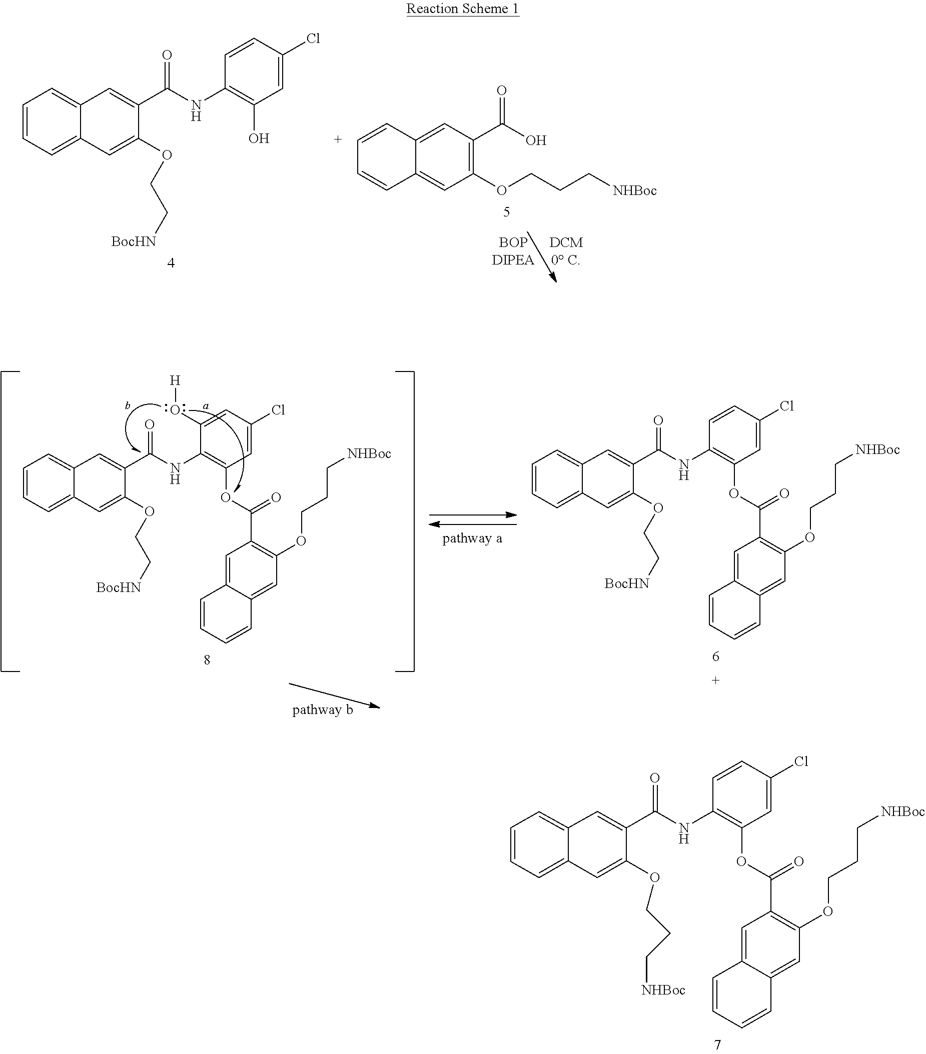

### Results and Discussion

The synthesis of compound 3 is presented in Scheme 1. Building blocks 4 (tert-butyl (2-((3-((4-chloro-2-hydroxyphenyl)carbamoyl)naphthalen-2-yl)oxy)ethyl)carbamate) and 5 (3-(3-((tert-butoxycarbonyl)amino)propoxy)-2-naphthoic acid) were prepared as described before.¹³ The ester coupling reaction between phenol 4 and acid 5 was found to be problematic. Under the coupling conditions we used before (BOP, DIPEA, room temperature),¹³ a mixture of compositional isomers 6 and 7 (2-(3-(3-((tert-butoxycarbonyl)amino)propoxy)-2-naphthamido)-5-chlorophenyl 3-(2-((tert-butoxy carbonyl)amino)ethoxy)-2-naphthoate) involving acyl group swapping was obtained. These two isomers were only partially separable by conventional silica gel column chromatograph. Other coupling conditions (EDCl, DIPEA; DCC, DIPEA; MSCl, Et₃N) were also attempted, but the results were found to be similar. The formation of the unanticipated alternative ester 7 was likely a result of generation of imide intermediate 8 (2-(3-(2-((tert-butoxycarbonyl)amino)ethoxy)-2-naphthamido)-5-chloro-3-hydroxyphenyl 3-(3-((tert-butoxycarbonyl) amino)propoxy)-2-naphthoate) although it was not detected from the reaction mixture. The intermediate 8 could be generated either before the formation of 6 (2-(3-(2-((tert-butoxycarbonyl) amino)ethoxy)-2-naphthamido)-5-chlorophenyl 3-(3-((tert-butoxycarbonyl)amino) propoxy)-2-naphthoate) or after 6 was formed. Once 8 was produced, it could generate either 6 or 7 through an ester bond formation (Scheme 1). After considerable experimentation involving the modifications of solvents, coupling reagents and temperature, we found that lowering the reaction temperature from room temperature to 0° C. and shortening the reaction time to 1 h could significantly inhibit the formation of 7 although complete reaction conversion was not achieved under these conditions. The Boc groups (tert-butyloxycarbonyl protecting groups) in 6 were then deprotected under acidic condition (HCl, DCM) to give 3 uneventfully. As expected, compound 3 was found to be highly soluble in ddlH₂O (>100 mg/mL). This is in strong contrast to 666-15, which is much less soluble in ddlH₂O (<0.5 mg/mL).

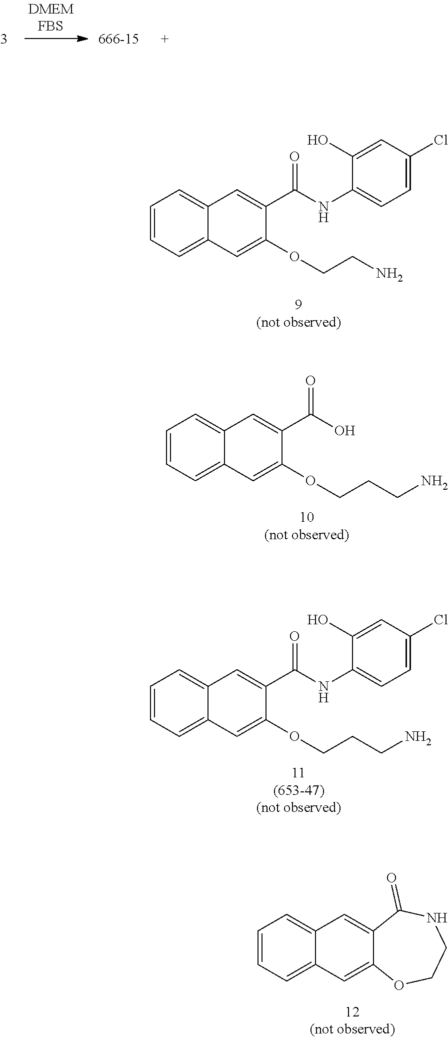 [SMILES: NCCOc1cc2ccccc2cc1C(=O)Nc1ccc(Cl)cc1O ; NCCCOc1cc2ccccc2cc1C(=O)O ; NCCCOc1cc2ccccc2cc1C(=O)Nc1ccc(Cl)cc1O ; O=C1NCCOc2cc3ccccc3cc21]

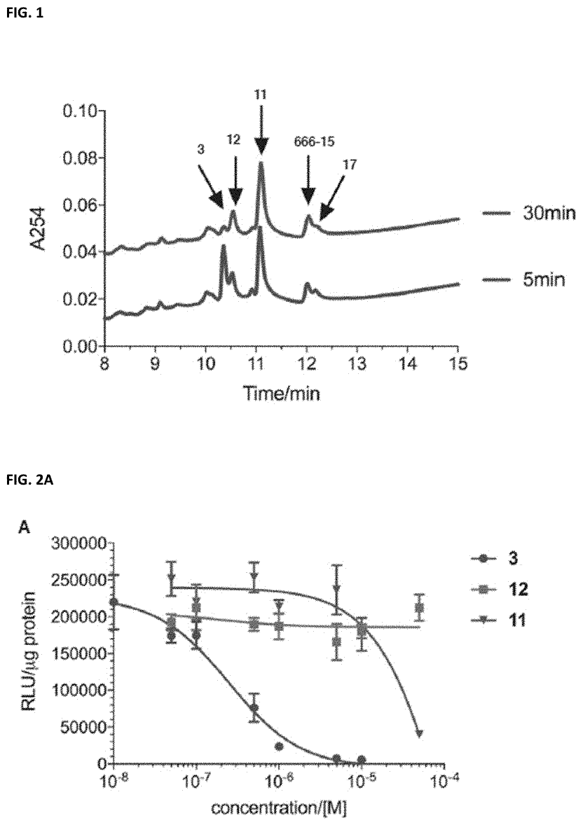

In the table above, Compound 3 was converted into 666-15, 11 (3-(3-aminopropoxy)-N-(4-chloro-2-hydroxyphenyl)-2-naphthamide) and 12 (3,4-dihydronaphtho[2,3-f][1,4]oxazepin-5(2H)-one) in the complete cell culture media. (A) Reaction conversion of 3 in complete cell culture media to generate a mixture of 666-15, 11 and 12. HPLC chromatograms of compound 3 incubated in complete cell culture media for 5 and 30 minutes are represented in FIG. 1.

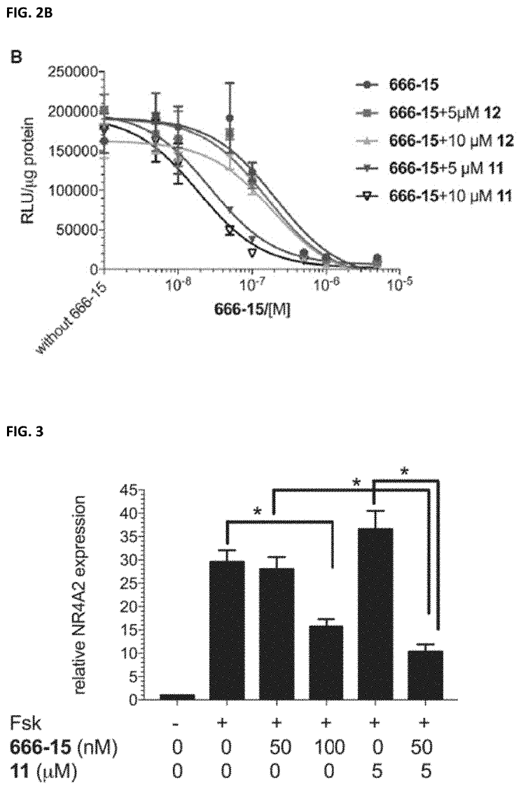

With the desired compound 3 in hand, we first investigated the possibility of transformation of 3 into 666-15 in the complete cell culture media (Dulbecco's Modified Eagle Medium with 10% fetal bovine serum (FBS)). When 3 was incubated with the complete cell culture media at 37° C., it was rapidly converted into four new product peaks as assessed by HPLC (FIG. 2B). In stark contrast to the structural congener 2,¹⁵ only a small amount of 666-15 was generated under the reaction condition. In order to investigate if simple ester hydrolysis was occurring to generate products 9 and 10 (FIG. 2A), we compared the main peaks eluted at 10.5 min and 11.2 min with standard samples of 9 and 10 by HPLC. However, these peaks did not correspond to either compound 9 or 10 (data not shown).

To identify the products formed from incubating 3 in the complete cell culture media, we treated compound 3 in phosphate-buffered saline (PBS, pH=7.40) (Scheme 2). An additional Boc protection step was added to facilitate purification of compounds containing free amino groups. After careful silica gel column purification and NMR analyses, we obtained an inseparable mixture of 13 (tert-butyl (3-((3-((2-((3-((2-((tert-butoxycarbonyl)oxy)-4-chlorophenyl)carbamoyl)naphthalen-2-yl)oxy)ethyl)carbamoyl) naphthalen-2-yl)oxy)propyl)carbamate) and 14 (tert-butyl (2-((3-((3-((3-((2-((tert-butoxycarbonyl)oxy)-4-chlorophenyl)carbamoyl)naphthalen-2-yl)oxy)propyl)carbamoyl)naphthalen-2-yl)oxy)ethyl)carbamate) in 18% total yield with a ratio of 3:1 as assessed by ¹H NMR. Furthermore, two unexpected products 15 (tert-butyl (3-((3-((2-((tert-butoxycarbonyl)oxy)-4-chlorophenyl)carbamoyl)naphthalen-2-yl)oxy)propyl) carbamate)(47%) and 12 (29%) were formed. The identity of 12 was further confirmed by an independent synthesis from 16 (methyl 3-(2-((tert-butoxycarbonyl)amino)ethoxy)-2-naphthoate)′ through Boc deprotection and cyclization (Scheme 2).

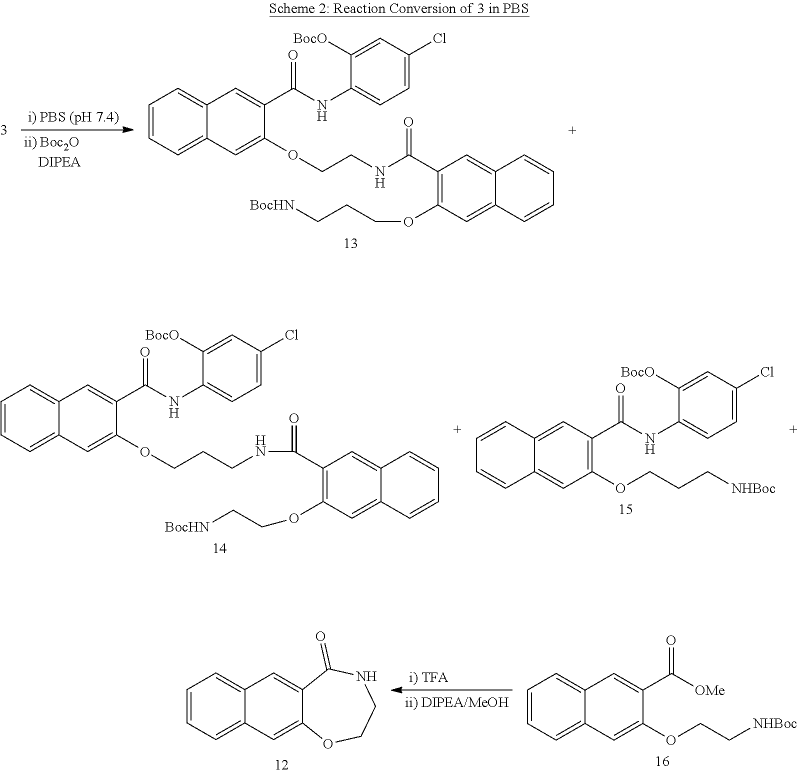

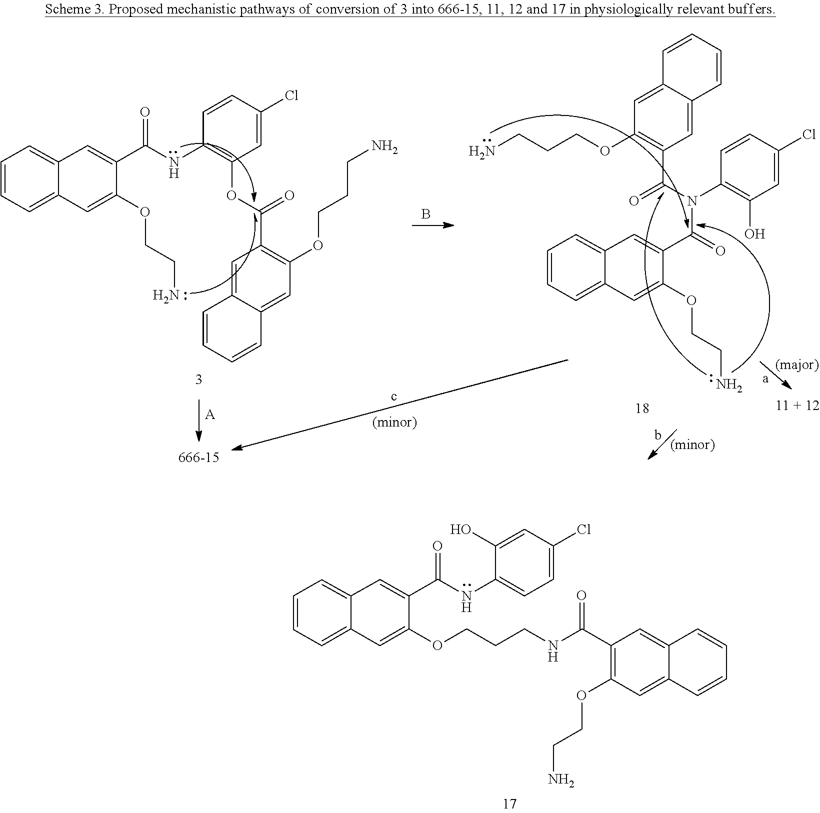

With the products identified from Scheme 2 and standard samples from Boc-deprotected products, we were able to assign the peak eluted at 10.5 min being 12, 11.2 min being 11 and the shoulder peak close to 666-15 being 17 (3-(2-aminoethoxy)-N-(3-((3-((4-chloro-2-hydroxyphenyl) carbamoyl)naphthalen-2-yl)oxy)propyl)-2-naphthamide)(FIG. 2B and Scheme 3). Based on our previous results with compound 2,¹⁵ we initially hypothesized that the mechanism of formation of 666-15 from 3 would be through pathway A by a long-range O,N-acyl transfer reaction (Scheme 3). Although we could not completely rule out this possibility, a more likely scenario is through pathway B as shown in Scheme 3. In this mechanism, imide 18 (3-(2-aminoethoxy)-N-(3-(3-aminopropoxy)-2-naphthoyl)-N-(4-chloro-2-hydroxyphenyl)-2-naphthamide) was first formed from 3. Three possibilities exist for imide 18. Pathway a would generate 11 and 12 and this route was found to be the major pathway. Minor pathways b and c would generate 17 and 666-15, respectively.

TABLE 1
CREB-inhibitory activity of different compounds

| Compound | CREB inhibition (IC50, μM)^{a} |
|---|---|
| 666-15 | 0.081 ± 0.04 |
| 3 | 0.25 ± 0.11 |
| 11 | 26.3 ± 13.6 |
| 12 | >50 |

^{a} The IC₅₀ refers to the concentration needed to inhibit 50% of the CREB-mediated gene transcription in HEK 293T cells using a transcription renilla luciferase reporter assay. The data are presented as mean±standard deviation (SD) of at least two independent experiments, which were performed in triplicates.

Because generation of the potent CREB inhibitor 666-15 from 3 in the complete tissue culture media was not the major pathway (FIG. 2B), we expected that compound 3 would not be a potent CREB inhibitor. To test this possibility, we employed a transcription luciferase reporter assay in HEK 293T cells.²² Briefly, the cells were transfected with a CREB renilla luciferase reporter plasmid (CRE-RLuc). This plasmid expresses renilla luciferase under the control of three tandem copies of CRE in the promoter region. After transfection, the cells were treated with different concentrations of compound 3 for 30 min, when forskolin (Fsk) was added to stimulate CREB phosphorylation and its subsequent transcription activity. In this assay, 666-15 has an IC₅₀ ^{˜}80 nM (Table 1).¹³ Surprisingly, compound 3 was found to be quite potent with an IC₅₀=0.25±0.11 μM (Table 1 and FIG. 3A) despite the fact that only a small fraction of 3 was converted into 666-15 under these conditions (FIG. 2B). This result suggested that other components in the reaction mixture might inhibit CREB-mediated gene transcription. Thus, the major compounds 11 and 12 were separately evaluated for their CREB inhibition potency. Compound 11, was found to be a very weak inhibitor with IC₅₀=26.3±13.6 μM while compound 12 was inactive (Table 1 and FIG. 3A). Consistent with its weak activity in inhibiting CREB's transcription, compound 11 did not inhibit KIX-KID interaction as assessed in a split renilla luciferase complementation assay (IC50>50 μM) 22

The rather weak CREB inhibition activity associated with 11 and 12 suggested other possibilities to account for the potent CREB inhibition activity of 3. One of the possibilities was that the mixture might have a synergistic activity. To test this possibility, we evaluated the combination of 666-15 with 11 or 12. As shown in FIG. 3B, combination of 666-15 with 12 did not produce further inhibition of CREB-mediated gene transcription. However, when 666-15 was combined with 11, it significantly potentiated activity of 666-15 in inhibiting CREB-mediated gene transcription. Importantly, the synergistic inhibition occurred at concentrations where 11 alone was not inhibiting CREB-mediated gene transcription (FIGS. 3A-B). The synergy between 666-15 and 11 was further demonstrated by the low combination indexes (CI) calculated using Chou-Talalay method (Figure S2 and Table S1), where CI>1.0 indicates antagonism, CI=1.0 indicates additive effect and CI<1.0 indicates synergism.²³ These results indicated that the potent CREB inhibition activity of 3 is primarily a result of the combination of in situ generated 666-15 and 11.

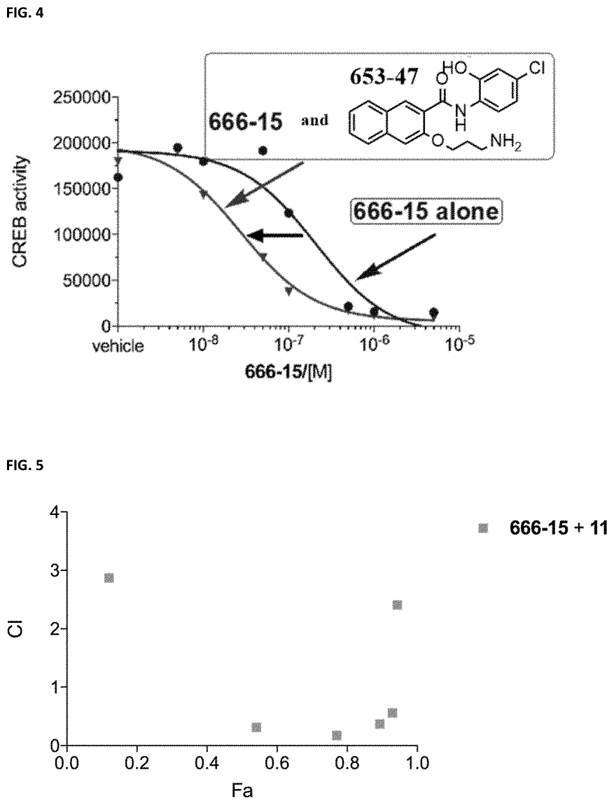

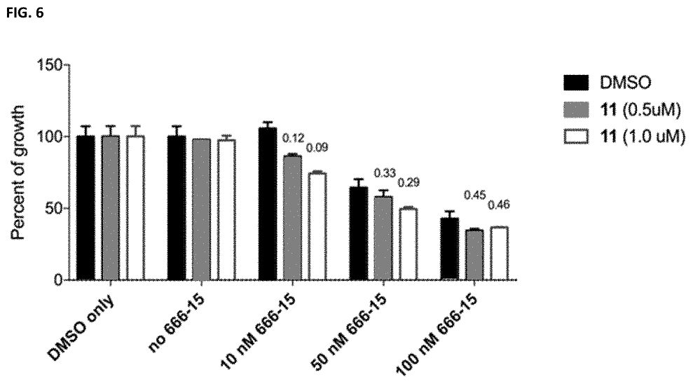

To further test that compound 11 could potentiate 666-15's CREB inhibitory activity, we evaluated the endogenous CREB target gene expression in HEK 293T cells. Nuclear Receptor Subfamily 4 Group A Member 2 (NR4A2) is a well-established CREB target gene in HEK 293T cells.²³ We have shown that NR4A2 was robustly stimulated by Fsk and its expression was suppressed by 666-15.¹³ Therefore, we investigated the expression level of NR4A2 in HEK293T cells with the combination of 666-15 and 11 using quantitative reverse transcription polymerase chain reaction (qRT-PCR). As reported previously,¹³ the expression level of NR4A2 was stimulated by Fsk (FIG. 4). When the cells were pretreated with low concentration of 666-15 (50 nM), the expression level of NR4A2 was not significantly reduced. At higher concentration (100 nM), its transcript level was significantly inhibited. Consistent with the transcription reporter assay results shown in FIG. 3B, 5 μM of compound 11 alone did not inhibit the expression of NR4A2. Instead, a minor increase of its expression was observed. However, when low concentration of 666-15 (50 nM) was combined with compound 11 (5 μM), significant inhibition of NR4A2 transcription was observed (FIG. 4). Together with the transcription reporter assay results shown in FIG. 3B, these data support that while compound 11, which was named as 653-47, was not active alone, it could significantly potentiate the CREB inhibitory activity of 666-15.

We previously showed that 666-15 potently inhibited breast cancer cell growth.¹³ Our discovery of 11 as a potentiator to enhance 666-15's potency in inhibiting CREB-mediated gene transcription suggested that it might also potentiate 666-15's anticancer potential in breast cancer cells. To test this hypothesis, we treated MDA-MB-231 cells with different concentrations of 666-15 along with or without 11 for 72 h. Then the antiproliferative effect was evaluated using MTT assay as described before.¹³ As shown in Figure S3, 666-15 was able to dose-dependently inhibit the cell growth. Compound 11 alone did not appreciably inhibit the cell growth at low concentrations (0.5 or 1.0 μM). However, when 11 was combined with 666-15, it further enhanced 666-15's anti-proliferative effect as indicated by the CI values. The CI values are all less than 1.0, indicating synergism between 666-15 and 11.

Inhibition of CREB-mediated gene transcription. HEK 293T cells were transfected with CRE-RLuc. Then the cells were treated with increasing concentrations of individual compounds (FIG. 2A) or drug combinations (FIG. 2B) for 30 min before the addition of Fsk (10 μM) for 5 h. The renilla luciferase activity was normalized to the protein concentration in each well and expressed as relative luciferase unit (RLU)/μg protein. The errors are SEM from one representative experiment in triplicate.

FIG. 3 represents synergistic inhibition of CREB-mediated gene transcription in HEK 293T cells by 666-15 and 11. HEK 293T cells were treated with 666-15 and 11 for 1 h, when the cells were stimulated with Fsk (10 μM) for 45 min. Then the cells were subjected to qRT-PCR analysis after the total RNA was isolated using HPRT as a reference gene. The errors are SEM of two independent experiments performed in triplicates. * P<0.05 by student t-test.

We describe herein our unexpected discovery of a potentiator compound 11 or 653-47 to synergistically inhibit CREB-mediated gene transcription with 666-15. We initially set out to improve the aqueous solubility of 666-15 by designing an ester prodrug 3, which was envisioned to undergo a long-range O,N-acyl transfer reaction. Our results showed that a modified O,N-acyl transfer process through an imide intermediate is a more likely mechanism of formation of 666-15 from the ester prodrug 3 (Scheme 3). Unexpectedly, we discovered compound 653-47 as an enhancer of 666-15's activity in inhibiting CREB-mediated gene transcription and breast cancer cell growth even though 653-47 alone does not inhibit these activities. This discovery opens new avenues to further target CREB-mediated gene transcription with small molecules. This discovery of 653-47 also provides a unique tool to study CREB regulation.

Methods of Use

Provided herein is a method of enhancing the effect of an anti-cancer agent in a subject, the method comprising administering to the subject in need thereof a therapeutically effective amount of an anti-cancer agent and a therapeutically effective amount of a compound of Formula (I):

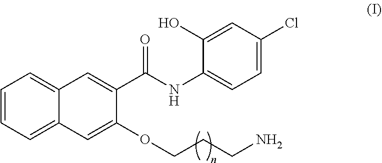

wherein n is an integer selected from the group of 0, 1, 2, 3, and 4;

or a pharmaceutically acceptable salt thereof.

In some embodiments, the anti-cancer agent is an agent known for the treatment of one or more cancers or malignant neoplasms selected from the group of lung cancer (including non-small cell lung cancer), prostate cancer, ovarian cancer, cervical cancer, breast cancer (including triple negative breast cancer), melanoma, leukemia (including acute myeloid leukemia), liver cancer, thyroid cancer, uterine cancer, bladder cancer, bone cancer, colon cancer, central nervous system cancer, esophageal cancer, gall bladder cancer, gastrointestinal cancer, head and neck cancer, Hodgkin's Disease, non-Hodgkin's lymphomas, laryngeal cancer, neuroblastoma, pancreatic cancer, rectal cancer, renal cancer, retinoblastoma, stomach cancer, testicular cancer, myeloma, tonsil cancer, Wilms' tumor or a combination thereof. In some embodiments, the cancer or malignant neoplasm associated with or dependent upon CREB-mediated gene transcription is selected from this same group. The methods, combinations, and pharmaceutical compositions herein may be used for primary or secondary (metastatic) cancers

Provided herein is a method of enhancing the effect of an anti-cancer agent in a subject, the method comprising administering to the subject in need thereof a therapeutically effective amount of an inhibitor of CREB-mediated gene transcription and a therapeutically effective amount of a compound of Formula (I):

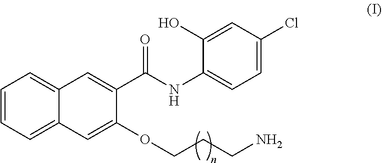

wherein n is an integer selected from the group of 0, 1, 2, 3, and 4, or a pharmaceutically acceptable salt thereof.

Provided herein is a method of treatment in a subject of a cancer or malignant neoplasm associated with or dependent upon CREB-mediated gene transcription, the method comprising administering to the subject in need thereof a therapeutically effective amount of a compound of the formula:

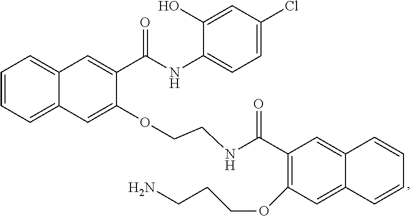 [SMILES: NCCCOc1cc2ccccc2cc1C(=O)NCCOc1cc2ccccc2cc1C(=O)Nc1ccc(Cl)cc1O]

or a pharmaceutically acceptable salt thereof;

and a therapeutically effective amount of a compound of Formula (I):

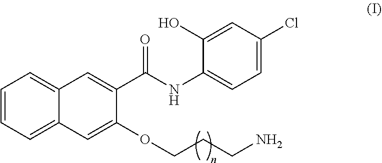

wherein n is an integer selected from the group of 0, 1, 2, 3, and 4, or a pharmaceutically acceptable salt thereof.

Also provided herein is a pharmaceutical composition comprising a therapeutically effective amount of a compound of the formula:

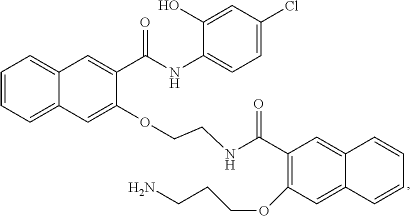 [SMILES: NCCCOc1cc2ccccc2cc1C(=O)NCCOc1cc2ccccc2cc1C(=O)Nc1ccc(Cl)cc1O]

or a pharmaceutically acceptable salt thereof;

and a therapeutically or synergistically effective amount of a compound of Formula (I):

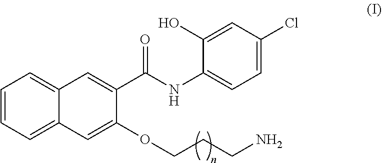

wherein n is an integer selected from the group of 0, 1, 2, 3, and 4, or a pharmaceutically acceptable salt thereof.

The methods herein may be used in treatments of one or more cancers selected from the group of lung cancer (including non-small cell lung cancer), prostate cancer, ovarian cancer, cervical cancer, breast cancer (including triple negative breast cancer), melanoma, leukemia (including acute myeloid leukemia), liver cancer, thyroid cancer, uterine cancer, bladder cancer, bone cancer, colon cancer, central nervous system cancer, esophageal cancer, gall bladder cancer, gastrointestinal cancer, head and neck cancer, Hodgkin's Disease, non-Hodgkin's lymphomas, laryngeal cancer, neuroblastoma, pancreatic cancer, rectal cancer, renal cancer, retinoblastoma, stomach cancer, testicular cancer, myeloma, tonsil cancer, Wilms' tumor or a combination thereof.

Provided herein is the compound of Formula (I):

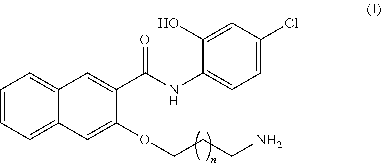

wherein n is an integer selected from the group of 0, 1, 2, 3, and 4, or a pharmaceutically acceptable salt thereof, for use in a method of enhancing the effect of an anti-cancer agent in a subject.

Provided herein is the compound of Formula (I):

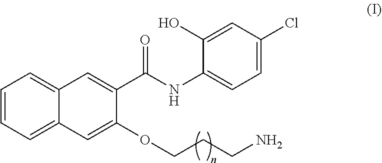

wherein n is an integer selected from the group of 0, 1, 2, 3, and 4, or a pharmaceutically acceptable salt thereof, for use in a method of enhancing the effect of an inhibitor or antagonist of CREB-mediated gene expression in a subject.

Provided herein is the use of a compound of Formula (I):

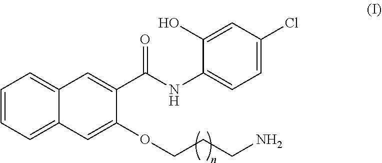

wherein n is an integer selected from the group of 0, 1, 2, 3, and 4, or a pharmaceutically acceptable salt thereof, in the preparation of a medicament for use in a method of enhancing the effect of an anti-cancer agent in a subject.

Provided herein is the use of a compound of Formula (I):

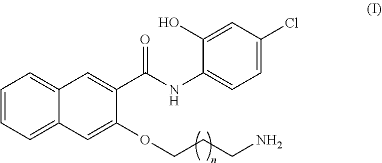

wherein n is an integer selected from the group of 0, 1, 2, 3, and 4, or a pharmaceutically acceptable salt thereof, in the preparation of a medicament for use in a method of enhancing the effect of an inhibitor or antagonist of CREB-mediated gene expression in a subject.

Provided herein is a compound of Formula (I):

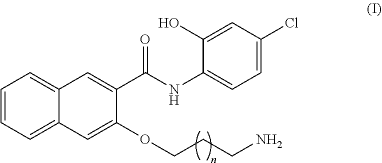

wherein n is an integer selected from the group of 0, 1, 2, 3, and 4, or a pharmaceutically acceptable salt thereof, for use in a method of enhancing the effect of an anti-cancer agent in a subject.

Provided herein is a compound of Formula (I):

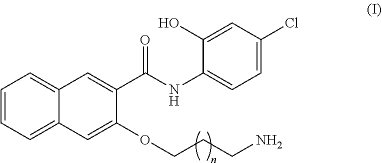

wherein n is an integer selected from the group of 0, 1, 2, 3, and 4, or a pharmaceutically acceptable salt thereof for use in a method of enhancing the effect of an inhibitor or antagonist of CREB-mediated gene expression in a subject.

For each of embodiments comprising a use, method, and composition herein comprising a compound of Formula (I), or a pharmaceutically acceptable salt thereof, or the use thereof, there is a further embodiment for the same use, method, or composition in which the integer n defining the compound of Formula (I), or a pharmaceutically acceptable salt thereof, is selected from the group of 0, 1, and 2.

For each of embodiments comprising a use, method, and composition herein comprising a compound of Formula (I), or a pharmaceutically acceptable salt thereof, or the use thereof, there is a further embodiment for the same use, method, or composition in which the integer n defining the compound of Formula (I), or a pharmaceutically acceptable salt thereof, is selected from the group of 0 and 1.

For each of embodiments comprising a use, method, and composition herein comprising a compound of Formula (I), or a pharmaceutically acceptable salt thereof, or the use thereof, there is a further embodiment for the same use, method, or composition in which the compound of Formula (I) has the structure:

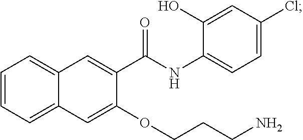 [SMILES: NCCCOc1cc2ccccc2cc1C(=O)Nc1ccc(Cl)cc1O]

or a pharmaceutically acceptable salt thereof.

For each of embodiments comprising a use, method, and composition herein comprising a compound of Formula (I), or a pharmaceutically acceptable salt thereof, or the use thereof, there is a further embodiment for the same use, method, or composition in which the compound of Formula (I) has the structure:

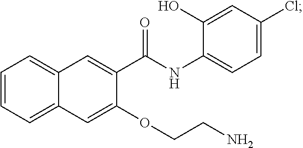 [SMILES: NCCOc1cc2ccccc2cc1C(=O)Nc1ccc(Cl)cc1O]

or a pharmaceutically acceptable salt thereof.

An embodiment provides a method of treatment of enhancing the effect of an anti-cancer agent in a subject, the method comprising administering to the subject in need thereof a therapeutically effective amount of an anti-cancer agent and a therapeutically effective amount of a compound of Formula (II):

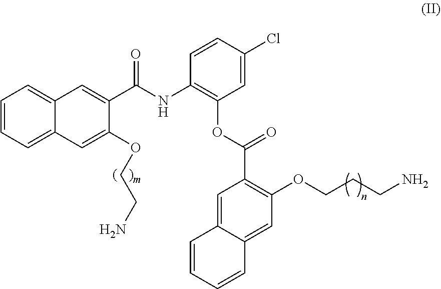

wherein:

m is an integer selected from the group of 0, 1, 2, 3, and 4;

n is an integer selected from the group of 0, 1, 2, 3, and 4;

or a pharmaceutically acceptable salt thereof.

Another embodiment provides a method of treatment in a subject of a cancer or malignant neoplasm associated with or dependent upon CREB-mediated gene transcription, the method comprising administering to the subject in need thereof a therapeutically effective amount of an anti-cancer agent and a therapeutically effective amount of a compound of Formula (II):

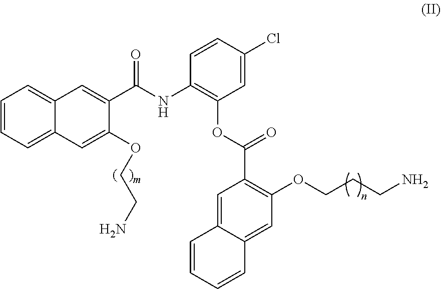

wherein:

m is an integer selected from the group of 0, 1, 2, 3, and 4;

n is an integer selected from the group of 0, 1, 2, 3, and 4;

or a pharmaceutically acceptable salt thereof.

A compound of Formula (II), or a pharmaceutically acceptable salt thereof, may be used in the methods described above for each of the cancers and with each of the anticancer agents described herein for the compound of Formula (I) and 666-15, particularly a cancer or malignant neoplasm associated with or dependent upon CREB-mediated gene transcription.

In some embodiments, the cancer or malignant neoplasm associated with or dependent upon CREB-mediated gene transcription is non-small cell lung cancer.

In other embodiments, the cancer or malignant neoplasm associated with or dependent upon CREB-mediated gene transcription is acute myeloid leukemia.

Another embodiment provides a pharmaceutical composition comprising a pharmaceutically acceptable carrier or excipient and a therapeutically effective amount of an anti-cancer agent and a therapeutically effective amount of a compound of Formula (II):

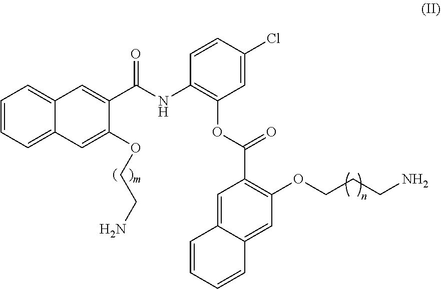

wherein:

m is an integer selected from the group of 0, 1, 2, 3, and 4;

n is an integer selected from the group of 0, 1, 2, 3, and 4;

or a pharmaceutically acceptable salt thereof.

For each of the embodiments, methods of treatment, and pharmaceutical compositions above concerning the use of a compound of Formula (II), or a pharmaceutically acceptable salt thereof, there is a further embodiment comprising the method for the cited use, or a further pharmaceutical composition, wherein m is an integer selected from the group of 0, 1, 2, and 3. For each of the methods and compositions above concerning the use of a compound of Formula (II), or a pharmaceutically acceptable salt thereof, there is also a further embodiment comprising the method and composition for the cited use wherein m is an integer selected from the group of 0, 1, and 2. For each of the methods or pharmaceutical compositions above concerning the use of a compound of Formula (II), or a pharmaceutically acceptable salt thereof, there is another embodiment for the cited method or composition wherein m is an integer selected from the group of 1 and 2.

For each of the methods and pharmaceutical compositions above concerning the use of a compound of Formula (II), or a pharmaceutically acceptable salt thereof, there is another embodiment comprising the method or composition for the cited use wherein m is 0. For each of the methods and pharmaceutical compositions above concerning the use of a compound of Formula (II), or a pharmaceutically acceptable salt thereof, there is yet another embodiment comprising the method or composition wherein m is 1. For each of the methods and pharmaceutical compositions above concerning the use of a compound of Formula (II), or a pharmaceutically acceptable salt thereof, there is still another embodiment comprising the method or composition wherein m is 2.

For each of the embodiments, methods, and pharmaceutical compositions above concerning the use of a compound of Formula (II), or a pharmaceutically acceptable salt thereof, there is a further embodiment comprising the method or composition wherein n is an integer selected from the group of 0, 1, 2, and 3. For each of the methods and pharmaceutical compositions above concerning the use of a compound of Formula (II), or a pharmaceutically acceptable salt thereof, there is also a further embodiment comprising the method or composition wherein n is an integer selected from the group of 0, 1, and 2. For each of the methods and pharmaceutical compositions above concerning the use of a compound of Formula (II), or a pharmaceutically acceptable salt thereof, there is another embodiment comprising the method or composition wherein n is an integer selected from the group of 1 and 2.

For each of the embodiments, methods, and pharmaceutical compositions above concerning the use of a compound of Formula (II), or a pharmaceutically acceptable salt thereof, there is another embodiment comprising the method or pharmaceutical composition wherein n is 0. For each of the methods above concerning the use of a compound of Formula (II), or a pharmaceutically acceptable salt thereof, there is yet another embodiment comprising the method or pharmaceutical composition wherein n k 1. For each of the embodiment comprising the methods or pharmaceutical compositions above concerning the use of a compound of Formula (II), or a pharmaceutically acceptable salt thereof, there is still another embodiment comprising the method or pharmaceutical composition wherein n is 2.

It is understood that the definitions of integers m and n above refer to each other in all possible combinations for a compound of Formula (II). For instance, in one embodiment both m and n are independently selected from the group of 0, 1, 2, 3, and 4. In another embodiment both m and n are independently selected from the group of 0, 1, 2, and 3. Other non-limiting embodiments examples are those in which m=1/n=0-4; m=3/n=1, 2 or 3; m=0-3/n=2; and m=1/n=1.

In some embodiments, the pharmaceutical composition above comprising a therapeutically effective amount of a compound of Formula (II), or a pharmaceutically acceptable salt thereof, is an enteric coated formulation.

Also provided is the use of a compound of Formula (II), or a pharmaceutically acceptable salt thereof, in the preparation of a medicament.

Pharmaceutical dosage forms such as tablets and the like commonly consist of a core containing one or more pharmacologically active ingredients together with various excipients which serve as binding agents, disintegrants, etc. The core may be provided with some form of a coating which may serve a variety of purposes such as rendering the dosage more palatable, improving the appearance, controlling release of the active ingredient both as to time and place, and/or for ease of identification. Coatings which are insoluble in the gastric juices of the stomach but which dissolve in the alkaline environment of the intestines are known and are needed fora variety of medical reasons not germane to the present invention. Such coatings are variously referred to as enteric coatings or enterosoluble coatings and will be so referred to hereinafter.

There are a number of known enteric materials, the most widely used probably being cellulose acetate phthalate and it is with this polymer that the present invention is particularly concerned. While being water-insoluble under low pH acidic conditions such as normally encountered in the human stomach, cellulose acetate phthalate is readily soluble in the higher pH environment of intestinal juices. Cellulose acetate phthalate is also readily soluble in volatile organic solvents such as acetone, and mixtures of acetone and methanol, acetone and methylene chloride, etc. and until fairly recently coating compositions were formed by dissolving the polymer in organic solvent. This practice of forming a coating composition by dissolving a polymer in organic solvent has also been used with water-insoluble non-enteric polymers such as ethyl cellulose. To the solution are frequently added pigments, surfactants, plasticizers, preservatives, flavors, etc. and the final composition is sprayed or otherwise applied to the dosage form so as to provide a continuous film upon evaporation of the solvent.

Other enteric coatings known in the art include those that cellulose based, methacrylate-based, polyvinyl acetate phthalate-based, cellulose acetate phthalate-based, shellac-based, Methyl methacrylate-methacrylic acid copolymer-based, cellulose acetate trimellitate-based, hydroxypropyl methyl cellulose phthalate-based (HPMCP-based), polyvinyl acetate phthalate-based, hydroxyethyl ethyl cellulose phthalate-based, cellulose acetate tetrahydrophthalate-based, cellulose acetate phthalate-based, cellulose acetate succinate-based, hydroxypropyl methyl cellulose acetate succinate-based (hypromellose acetate succinate), diethyl phthalate-based, acrylic resin-based, as well as those based on shellac and wax compositions. Others comprise a copolymer of methacrylic acid and ethyl acrylate.

### Definitions

The term “subject” includes both human and veterinary subjects.

A “therapeutically effective amount”, “pharmaceutically effective amount” or “diagnostically effective amount” refers to a quantity of a specified agent sufficient to achieve a desired effect in a subject being treated with that agent. For example, this may be the amount of a compound disclosed herein useful in detecting or treating thyroid cancer in a subject. Ideally, a therapeutically effective amount or diagnostically effective amount of an agent is an amount sufficient to inhibit or treat the disease without causing a substantial cytotoxic effect in the subject. The therapeutically effective amount or diagnostically effective amount of an agent will be dependent on the subject being treated, the severity of the affliction, and the manner of administration of the therapeutic composition.

In regard to compounds of Formula (I), or a pharmaceutically acceptable salt thereof, and a therapeutically or pharmaceutically effective amount may also be a “synergistically effective amount”, i.e. the amount of a compound of Formula (I), or a pharmaceutically acceptable salt thereof, required to enhance or allow the activity of an additional cancer treatment or oncology agent, such as an inhibitor or antagonist of CREB-mediated gene expression. The same definition of “synergistically effective amount” and all other definitions herein in regard to a compound of Formula (I), or a pharmaceutically acceptable salt thereof, also applies to a compound of Formula (II), or a pharmaceutically acceptable salt thereof.

“Cancer” or “malignant neoplasm” includes a neoplasm that has undergone characteristic anaplasia with loss of differentiation, increased rate of growth, invasion of surrounding tissue, and which is capable of metastasis.

“Tumor” refers to a mass of cells resulting from excessive cellular multiplication. A tumor is a neoplasm that may be either malignant or non-malignant (benign) and includes both solid and non-solid tumors (such as hematologic malignancies). As used herein, this term also encompasses other cell types found in the tumor microenvironment, such as vascular endothelial cells, pericytes, fibroblasts and/or other stromal elements.

A “prodrug” is an active or inactive compound that is modified chemically through in viva physiological action, such as hydrolysis, metabolism and the like, into an active compound following administration of the prodrug to a subject. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985).

Pharmaceutically acceptable prodrugs refer to compounds that are metabolized, for example, hydrolyzed or oxidized, in the subject to form an agonist compound of the present disclosure, Typical examples of prodrugs include compounds that have one or more biologically labile protecting groups on or otherwise blocking a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The term “prodrug” is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

“Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The compounds disclosed herein may be included in pharmaceutical compositions (including therapeutic and prophylactic formulations); typically combined together with one or more pharmaceutically acceptable vehicles or carriers and; optionally; other therapeutic ingredients (for example, antibiotics or anti-inflammatories). The compositions disclosed herein may be advantageously combined and/or used in combination with other antiproliferative therapeutic agents, different from the subject compounds. In many instances, co-administration in conjunction with the subject compositions will enhance the efficacy of such agents. Exemplary antiproliferative agents include cyclophosphamide, methotrexate, adriamycin, cisplatin, daunomycin, vincristine, vinblastine, vinarelbine, paclitaxel, docetaxel, tamoxifen, flutamide, hydroxyurea, and mixtures thereof.

Such pharmaceutical compositions can be administered to subjects by a variety of mucosal administration modes, including by oral, rectal, intranasal, intrapulmonary, or transdermal delivery, or by topical delivery to other surfaces. Optionally; the compositions can be administered by non-mucosal routes, including by intramuscular, subcutaneous, intravenous, intra-arterial, intra-articular, intraperitoneal, intrathecal, intracerebroventricular, or parenteral routes. In other alternative embodiments, the compound can be administered ex vivo by direct exposure to cells, tissues or organs originating from a subject.

To formulate the pharmaceutical compositions, the compound can be combined with various pharmaceutically acceptable additives, as well as a base or vehicle for dispersion of the compound, Desired additives include, but are not limited to, pH control agents, such as arginine, sodium hydroxide, glycine, hydrochloric add, citric add, and the like. In addition, local anesthetics (for example, benzyl alcohol), isotonizing agents (for example, sodium chloride, mannitol, sorbitol), adsorption inhibitors (for example, Tween 80), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The compound can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl(meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, polylactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The compound can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent, Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages, A number of these are known in the art to be biodegradable and to lead to degradation products having no or low toxicity. Exemplary polymers include polyglycolic adds and polylactic adds, poly(DL-lactic acid-co-glycolic acid), poly(D-lactic acid-co-glycolic acid), and poly(L-lactic acid-co-glycolic add). Other useful biodegradable or bioerodible polymers include, but are not limited to, such polymers as poly(epsilon-caprolactone), poly(epsilon-aprolactone-CO-lactic add), poly(epsilon.-aprolactone-CO-glycolic acid), poly(beta-hydroxy butyric add), poly(alkyl-2-cyanoacrilate), hydrogels, such as poly(hydroxyethyl methacrylate), polyamides, poly(amino adds) (for example, L-leucine, glutamic acid, L-aspartic acid and the like), polyester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and controlled Release Drug Delivery Systems, J. R. Robinson, cd., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture; storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In accordance with the various treatment methods of the disclosure, the compound can be delivered to a subject in a manner consistent with conventional methodologies associated with management of the disorder for which treatment or prevention is sought. In accordance with the disclosure herein, a prophylactically or therapeutically effective amount of the compound and/or other biologically active agent is administered to a subject in need of such treatment for a time and under conditions sufficient to prevent, inhibit, and/or ameliorate a selected disease or condition or one or more symptom(s) thereof.

Typical subjects intended for treatment with the compositions and methods of the present disclosure include humans, as well as non-human primates and other animals. To identify subjects for prophylaxis or treatment according to the methods of the disclosure, accepted screening methods are employed to determine risk factors associated with a targeted or suspected disease of condition (for example, CREB-mediated cancer) as discussed herein, or to determine the status of an existing disease or condition in a subject. These screening methods include, for example, diagnostic methods, such as various ELISA, western blot, immunohistochemical analysis, immunofluorescence staining, and real time RT-PCR analysis, which are available and well known in the art to detect and/or characterize disease-associated markers. These and other routine methods allow the clinician to select patients in need of therapy using the methods and pharmaceutical compositions of the disclosure.

The administration of the compound of the disclosure can be for either prophylactic or therapeutic purpose. When provided prophylactically, the compound is provided in advance of any symptom. The prophylactic administration of the compound serves to prevent or ameliorate any subsequent disease process. When provided therapeutically, the compound is provided at (or shortly after) the onset of a symptom of disease or infection.

For prophylactic and therapeutic purposes, the compound can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the compound (for example, amounts that are effective to elicit a desired immune response or alleviate one or more symptoms of a targeted disease). In alternative embodiments, an effective amount or effective dose of the compound may simply inhibit or enhance one or more selected biological activities correlated with a disease or condition, as set forth herein, for either therapeutic or diagnostic purposes.

The actual dosage of the compound will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the compound for eliciting the desired activity or biological response in the subject. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects. A non-limiting range for a therapeutically effective amount of a compound and/or other biologically active agent within the methods and formulations of the disclosure is about 0.01 mg/kg body weight to about 10 mg/kg body weight, such as about 0.05 mg/kg body weight to about 5 mg/kg body weight, or about 0.2 mg/kg body weight to about 2 mg/kg body weight.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, or intranasal delivery versus intravenous or subcutaneous delivery, Dosage can also be adjusted based on the release rate of the administered formulation, for example, of an intrapulmonary spray versus powder, sustained release oral versus injected particulate or transdermal delivery formulations, and so forth. To achieve the same serum concentration level, for example, slow-release particles with a release rate of 5 nanomolar (under standard conditions) would be administered at about twice the dosage of particles with a release rate of 10 nanomolar.

The instant disclosure also includes kits, packages and multi-container units containing the herein described pharmaceutical compositions, active ingredients, and/or means for administering the same for use in the prevention and treatment of diseases and other conditions in mammalian subjects. Kits for diagnostic use are also provided. In one embodiment, these kits include a container or formulation that contains one or more of the conjugates described herein. In one example, this component is formulated in a pharmaceutical preparation for delivery to a subject.

The conjugate is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

Experimental Section

Chemistry-General.

Glass Contout solvent purification system was used to purify all the anhydrous solvents to be used for reactions. Melting points were determined in capillary tubes using Mel-Temp and are uncorrected. All ¹H and ¹³C NMR spectra were obtained in a Bruker Avance 400 MHz spectrometer using CDCl₃ or DMSO-d₆ as the solvent and the chemical shifts of the residual CHCl₃ (δ 7.24) or DMSO (δ 2.50) were taken as reference. Chemical shifts (6δ) are reported in parts per million (ppm), and the signals are described as brs (broad singlet), d (doublet), dd (doublet of doublet), td (triplet of doublet), m (multiplet), q (quartet), s (singlet) and t (triplet). Coupling constants (J values) are given in Hz. Silica gel flash chromatography was performed using 230-400 mesh silica gel (EMD). All reactions were monitored using thin-layer chromatography (TLC) on silica gel plates (EMD). Yields were of purified compounds. All final compounds for biological evaluations were confirmed to be of >95% purity based on reverse phase HPLC (Waters, Milford, Mass.) analysis using an XBridge C18 column (4.6×150 mm) and detected at 254 nm. The mobile phases for HPLC are water and acetonitrile, both of which contained 0.1% TFA. The mass spectra were obtained from a Thermo Electron LTQ-Orbitrap Discovery high resolution mass spectrometer (Thermo Scientific) with electrospray operated either in positive or negative mode.

### 2-(3-(2-Aminoethoxy)-2-naphthamido)-5-chlorophenyl 3-(3-Aminopropoxy)-2-naphthoate Dihydrochloride (3)

An HCl solution in Et₂O (2 M, 3 mL) was added to a stirred solution of 6 (75 mg, 0.095 mmol) in DCM (3 mL) at room temperature. The resulting mixture was stirred at room temperature for 4 hours. The solvent was removed and the residue was treated with acetone and the precipitate was collected by filtration to give product 3 (8 mg, 13%) as a white solid: m.p. 216-217° C. ¹H NMR (400 MHz, DMSO-d₆) δ 10.18 (s, 1H), 8.75 (s, 1H), 8.24 (brs, 3H), 8.09 (s, 1H), 8.01-7.78 (m, 8H), 7.66-7.60 (m, 3H), 7.58— 7.45 (m, 4H), 7.40 (t, J=7.6 Hz, 1H), 7.35 (t, J=7.7 Hz, 1H), 4.28 (t, J=5.4 Hz, 2H), 4.23 (t, J=5.4 Hz, 2H), 3.21 (q, J=5.8 Hz, 2H), 3.01 (q, J=5.8 Hz, 2H), 2.03 (quintet, J=6.0 Hz, 2H); HRESIMS Calcd for C₃₃H₃₁ClN₃O₅ [M+H]⁺ 584.1947. Found 584.1943.

### 2-(3-(2-((tert-Butoxycarbonyl)amino)ethoxy)-2-naphthamido)-5-chlorophenyl 3-(3-((tert-Butoxycarbonyl)amino)propoxy)-2-naphthoate (6)

To a stirred solution of 5¹⁵ (173 mg, 0.5 mmol) in dichloromethane (10 mL) was added BOP (221 mg, 0.5 mmol) and DIPEA (90 μL, 0.5 mmol). The reaction mixture was stirred at room temperature for 5 min. Then the reaction mixture was cooled down to 0° C. Compound 4¹³ (229 mg, 0.5 mmol) and another portion of DIPEA (120 μL, 0.65 mmol) were added sequentially. The resulting mixture was stirred for 1 hour at 0° C. The solvent was removed under reduced pressure and the residue was purified by flash column chromatography, eluting with dichloromethane-ethyl acetate (20:1) to give the crude product 6, which was further purified by the Biotage purification system, eluting with hexanes-ethyl acetate (20:1 to 4:1) to give pure product 6 (75 mg, 19%) and some less pure product (170 mg, 43%):¹H NMR (400 MHz, CDCl₃) δ 9.81 (s, 1H), 8.74 (s, 1H), 8.58 (s, 1H), 8.41 (d, J=8.8 Hz, 1H), 7.87 (d, J=8.2 Hz, 1H), 7.81 (d, J=8.2 Hz, 1H), 7.76 (d, J=8.2 Hz, 1H), 7.67 (d, J=8.2 Hz, 1H), 7.59 (t, J=7.6 Hz, 1H), 7.51 (t, J=7.5 Hz, 1H), 7.44-7.37 (m, 3H), 7.34 (dd, J=8.8, 2.3 Hz, 1H), 7.24 (s, 1H), 7.16 (s, 1H), 5.40 (brs, 1H), 4.73 (brs, 1H), 4.12 (t, J=7.1 Hz, 2H), 3.91 (t, J=5.4 Hz, 2H), 3.33-3.07 (m, 4H), 1.89 (brs, 2H), 1.35 (s, 9H), 1.33 (s, 9H); ¹³C NMR (101 MHz, CDCl₃) δ 163.67, 163.47, 156.28, 155.88, 155.05, 153.27, 142.16, 136.73, 135.79, 134.20, 129.69, 129.62, 129.58, 129.10, 128.98, 128.68, 128.35, 127.33, 126.70, 126.60, 126.38, 125.13, 125.03, 124.84, 122.73, 108.42, 107.92, 79.62, 78.88, 68.33, 67.55, 39.37, 38.30, 29.05, 28.36, 28.27.

### 3,4-Dihydronaphtho[2,3-f][1,4]oxazepin-5(2H)-one (12)

Compound 16 (103 mg, 0.3 mmol) was treated with TFA (1 mL) for 1 hour at room temperature. TFA was removed under reduced pressure and the residue was redissolved in MeOH (4 mL) and DIPEA (1 mL). The mixture was heated under reflux for overnight. The solvent was removed under reduced pressure and the residue was purified by flash column chromatography, eluting with dichloromethane-methanol (20:1) to give product 12 (25 mg, 39%) as a yellow solid: m.p. 128-129° C. ¹H NMR (400 MHz, CDCl₃) δ 8.39 (s, 1H), 7.91 (d, J=8.2 Hz, 1H), 7.78 (d, J=8.3 Hz, 1H), 7.56-7.51 (m, 1H), 7.50 (s, 1H), 7.45 (m, 1H), 7.07 (s, 1H), 4.39 (t, J=5.4 Hz, 2H), 3.48 (q, J=5.6 Hz, 2H); ¹³C NMR (101 MHz, CDCl₃) δ 171.14, 151.27, 136.06, 132.05, 130.11, 128.93, 128.09, 127.50, 126.81, 125.55, 118.36, 73.79, 40.50; HRESIMS Calcd for C₁₃H₁₂NO₂ [M+H]⁺ 214.0863. Found 214.0858.

Biology. General.

HEK293T cells were from ATCC and MDA-MB-231 cells were from the Development Therapeutics Program at the National Cancer Institute. The cells were routinely cultured in DMEM (Life Technologies) with 10% fetal bovine serum (FBS, Hyclone) in a humidified incubator with 5% CO₂ at 37° C. The cells were confirmed to be mycoplasma negative and authenticated through STR profiling. pCRE-RLuc was reported before.²² All the compounds for biological testing were dissolved in DMSO as stock solutions except compound 3, which was dissolved in DMF because compound 3 was found to be unstable in DMSO for long-term storage. Compounds 11 and 17 were reported previously.¹³

Inhibition of CREB-Mediated Gene Transcription.

HEK293T cells in a well of 6-well plate were transfected with a plasmid (1 μg) encoding renilla luciferase under the control of three copies of CRE (pCRE-RLuc) using Lipofectamin²⁰⁰⁰ (Life Technologies) for 3 h. Then the cells were replated into a 96-well plate and the cells were allowed to attach to the bottom of the plate for overnight. The cells were treated with different concentrations of the compounds for 30 min, when Fsk (10 μM) was added to the cells. The cells were further incubated for 5 h at 37° C. The media were removed and the cells were lysed in 30 μl 1× renilla luciferase lysis buffer (Promega) and the renilla luciferase activity was measured using the renilla luciferase assay system (Promega). The protein concentration in each well was determined using the Protein Assay Dye Reagent Concentrate (Bio-rad). The luciferase activity in each well was normalized to the protein concentration and expressed as relative luciferase unit (RLU)/μg protein. The IC₅₀ was calculated in Prism 5.0 using the non-linear regression analysis.

Conversion of Ester 3 in DMEM.

Compound 3 (200 μM) was incubated with complete tissue culture media (DMEM with 10% FBS) at 37° C. for various time periods. An aliquot of 10 μl was taken at a given time point and mixed with acetonitrile (90 μL). The mixture was centrifuged at 14,000×rpm for 5 min at room temperature in a tabletop centrifuge to precipitate proteins. The supernatant was collected and analyzed by HPLC eluting with a linear of water and acetonitrile, both of which contained 0.1% TFA.

Inhibition of Endogenous CREB Target Gene Expression.

HEK 293T cells in 6-well plates were treated with different concentrations of 666-15 along with or without 11 for 1 h. Then the cells were stimulated with Fsk (10 μM) for 45 min. The total RNA was isolated using NucleoSpin® RNA isolation kit (Takara Bio). Following RNA isolation, the first strand cDNA was synthesized using PrimeScript™ 1^{st} strand cDNA synthesis kit (Takara Bio) with random hexamers as the primers. Quantitative PCR reactions were performed with TB Green® Advantage® qPCR Premix (Takara Bio). The relative mRNA level was determined with double delta Cₜ method using HPRT as a reference gene. Primers used for qRT-PCR are available upon request.

Cell Growth Inhibition Assay.

The cells were plated in 96-well plates at 1,000 cells/well and allowed to attach to the bottom of the plate for overnight. Then the cells were treated with different concentrations of compounds for 72 h at 37° C. At the end of treatment, the viable cells were determined by incubating cells with MTT reagent (0.5 mg/mL) in the complete tissue culture media for 3 h. Then the media were removed and the formed purple formazan was dissolved in DMSO for quantification by absorbance at 570 nm using a SpectraMax i3 plate reader (Molecular Devices). The percent of growth is defined as 100×(A_{treated}−Aᵢₙᵢₜᵢₐₗ)/(A_{control}−Aᵢₙᵢₜᵢₐₗ), where A_{treated} represents absorbance in wells treated with a compound, Aᵢₙᵢₜᵢₐₗ represents the absorbance at time 0, and A_{control} denotes media-treated cells.

Statistics.

The statistics was computed using student t-test in Excel and P<0.05 was considered significant. The CI values were calculated using Chou-Talalay method implemented in CompuSyn.

The combination indexes (CI) of 666-15 and compounds 11 in inhibiting CREB-mediated gene transcription. The CI values were calculated using Chou-Talalay method.

| | 666-15 (μM) | | | | | |
|---|---|---|---|---|---|---|
| Compounds | 0.01 | 0.05 | 0.1 | 0.5 | 1.0 | 5.0 |
| 11 (5 μM) | 2.87 | 0.31 | 0.17 | 0.37 | 0.56 | 2.41 |

### REFERENCES

(1) Mayr, B.; Montminy, M. Transcriptional Regulation by the Phosphorylation-Dependent Factor Creb. Nat. Rev. Mol. Cell Biol. 2001, 2, 599-609.
(2) Cardinaux, J. R.; Notis, J. C.; Zhang, Q.; Vo, N.; Craig, J. C.; Fass, D. M.; Brennan, R. G.; Goodman, R. H. Recruitment of Creb Binding Protein Is Sufficient for Creb-Mediated Gene Activation. Mol. Cell. Biol. 2000, 20, 1546-1552.
(3) Radhakrishnan, I.; Perez-Alvarado, G. C.; Parker, D.; Dyson, H. J.; Montminy, M. R.; Wright, P. E. Solution Structure of the Kix Domain of Cbp Bound to the Transactivation Domain of Creb: A Model for Activator:Coactivator Interactions. Cell 1997, 91, 741-752.
(4) Hagiwara, M.; Alberts, A.; Brindle, P.; Meinkoth, J.; Feramisco, J.; Deng, T.; Karin, M.; Shenolikar, S.; Montminy, M. Transcriptional Attenuation Following Camp Induction Requires Pp-1-Mediated Dephosphorylation of Creb. Cell 1992, 70, 105-113.
(5) Wadzinski, B. E.; Wheat, W. H.; Jaspers, S.; Peruski, L. F.; Lickteig, R. L.; Johnson, G. L.; Klemm, D. J. Nuclear-Protein Phosphatase-2a Dephosphorylates Protein Kinase a Phosphorylated Creb and Regulates Creb Transcriptional Stimulation. Mol. Cell. Biol. 1993, 13, 2822-2834.
(6) Gu, T.; Zhang, Z.; Wang, J.; Guo, J.; Shen, W. H.; Yin, Y. Creb Is a Novel Nuclear Target of Pten Phosphatase. Cancer Res. 2011, 71, 2821-2825.
(7) Meeusen, B.; Janssens, V. Tumor Suppressive Protein Phosphatases in Human Cancer: Emerging Targets for Therapeutic Intervention and Tumor Stratification. Int. J. Biochem. Cell Biol. 2018, 96, 98-134.
(8) Xiao, X.; Li, B. X.; Mitton, B.; Ikeda, A.; Sakamoto, K. M. Targeting Creb for Cancer Therapy: Friend or Foe. Curr. Cancer Drug Targets 2010, 10, 384-391.
(9) Rodon, L.; Gonzalez-Junca, A.; Inda, M. D.; Sala-Hojman, A.; Martinez-Saez, E.; Seoane, J. Active Creb1 Promotes a Malignant Tgf-Beta2 Autocrine Loop in Glioblastoma. Cancer Discov. 2014, 4, 1230-1241.
(10) Zhuang, H.; Meng, X.; Li, Y.; Wang, X.; Huang, S.; Liu, K.; Hehir, M.; Fang, R.; Jiang, L.; Zhou, J. X.; Wang, P.; Ren, Y. Cyclic Amp Responsive Element-Binding Protein Promotes Renal Cell Carcinoma Proliferation Probably Via the Expression of Spindle and Kinetochore-Associated Protein 2. Oncotarget 2016, 7, 16325-16337.
(11) Johannessen, C. M.; Johnson, L. A.; Piccioni, F.; Townes, A.; Frederick, D. T.; Donahue, M. K.; Narayan, R.; Flaherty, K. T.; Wargo, J. A.; Root, D. E.; Garraway, L. A. A Melanocyte Lineage Program Confers Resistance to Map Kinase Pathway Inhibition. Nature 2013, 504, 138-142.
(12) Zhang, Y.; Zheng, D.; Zhou, T.; Song, H.; Hulsurkar, M.; Su, N.; Liu, Y.; Wang, Z.; Shao, L.; Ittmann, M.; Gleave, M.; Han, H.; Xu, F.; Liao, W.; Wang, H.; Li, W. Androgen Deprivation Promotes Neuroendocrine Differentiation and Angiogenesis through Creb-Ezh2-Tsp1 Pathway in Prostate Cancers. Nature communications 2018, 9, 4080.
(13) Xie, F.; Li, B. X.; Kassenbrock, A.; Xue, C.; Wang, X.; Qian, D. Z.; Sears, R. C.; Xiao, X. Identification of a Potent Inhibitor of Creb-Mediated Gene Transcription with Efficacious in Vivo Anticancer Activity. J. Med. Chem. 2015, 58, 5075-5087.
(14) Xie, F.; Li, B. X.; Xiao, X. Design, Synthesis and Biological Evaluation of Regioisomers of 666-15 as Inhibitors of Creb-Mediated Gene Transcription. Bioorg. Med. Chem. Lett. 2017, 27, 994-998.
(15) Li, B. X.; Xie, F.; Fan, Q.; Barnhart, K. M.; Moore, C. E.; Rheingold, A. L.; Xiao, X. Novel Type of Prodrug Activation through a Long-Range O,N-Acyl Transfer: A Case of Water-Soluble Creb Inhibitor. ACS Med. Chem. Lett. 2014, 5, 1104-1109.
(16) Li, B. X.; Gardner, R.; Xue, C.; Qian, D. Z.; Xie, F.; Thomas, G.; Kazmierczak, S. C.; Habecker, B. A.; Xiao, X. Systemic Inhibition of Creb Is Well-Tolerated in Vivo. Sci. Rep. 2016, 6, 34513.
(17) Kang, X.; Lu, Z.; Cui, C.; Deng, M.; Fan, Y.; Dong, B.; Han, X.; Xie, F.; Tyner, J. W.; Coligan, J. E.; Collins, R. H.; Xiao, X.; You, M. J.; Zhang, C. C. The Itim-Containing Receptor Lair1 Is Essential for Acute Myeloid Leukaemia Development. Nat. Cell Biol. 2015, 17, 665-677.
(18) Mantamadiotis, T. Towards Targeting Pi3k-Dependent Regulation of Gene Expression in Brain Cancer. Cancers (Basel) 2017, 9.
(19) Srinivasan, S.; Totiger, T.; Shi, C.; Castellanos, J.; Lamichhane, P.; Dosch, A. R.; Messaggio, F.; Kashikar, N.; Honnenahally, K.; Ban, Y.; Merchant, N. B.; VanSaun, M.; Nagathihalli, N. S. Tobacco Carcinogen-Induced Production of Gm-Csf Activates Creb to Promote Pancreatic Cancer. Cancer Res. 2018, 78, 6146-6158.
(20) Wiley, S. Z.; Sriram, K.; Liang, W.; Chang, S. E.; French, R.; McCann, T.; Sicklick, J.; Nishihara, H.; Lowy, A. M.; Insel, P. A. Gpr68, a Proton-Sensing Gper, Mediates Interaction of Cancer-Associated Fibroblasts and Cancer Cells. FASEB J. 2018, 32, 1170-1183.
(21) Rodriguez-Ubreva, J.; Catala-Moll, F.; Obermajer, N.; Alvarez-Errico, D.; Ramirez, R. N.; Company, C.; Vento-Tormo, R.; Moreno-Bueno, G.; Edwards, R. P.; Mortazavi, A.; Kalinski, P.; Ballestar, E. Prostaglandin E2 Leads to the Acquisition of Dnmt3a-Dependent Tolerogenic Functions in Human Myeloid-Derived Suppressor Cells. Cell reports 2017, 21, 154-167.
(22) Li, B. X.; Xiao, X. Discovery of a Small-Molecule Inhibitor of the Kix-Kid Interaction. Chembiochem 2009, 10, 2721-2724.
(23) Chou, T. C. Theoretical Basis, Experimental Design, and Computerized Simulation of Synergism and Antagonism in Drug Combination Studies. Pharmacol. Rev. 2006, 58, 621-681.
(24) Conkright, M. D.; Guzman, E.; Flechner, L.; Su, A. I.; Hogenesch, J. B.; Montminy, M. Genome-Wide Analysis of Creb Target Genes Reveals a Core Promoter Requirement for Camp Responsiveness. Mol. Cell 2003, 11, 1101-1108.

What is claimed:

1. A method of enhancing the effect of an anti-cancer agent in a subject, the method comprising administering to the subject in need thereof a therapeutically effective amount of an anti-cancer agent and a therapeutically effective amount of a compound of Formula (I): 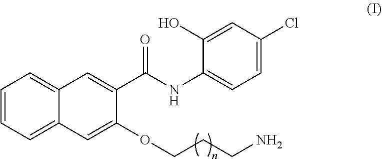
wherein n is an integer selected from the group of 0, 1, 2, 3, and 4, or a pharmaceutically acceptable salt thereof.

2. The method of claim 1, wherein the anti-cancer agent is an inhibitor of CREB-mediated gene transcription.

3. The method of claim 2, wherein the compound of Formula (I) is the compound: 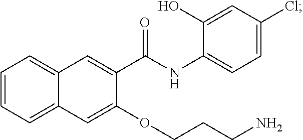 [SMILES: NCCCOc1cc2ccccc2cc1C(=O)Nc1ccc(Cl)cc1O]
or a pharmaceutically acceptable salt thereof.

4. The method of claim 2, wherein the compound of Formula (I) is the compound: 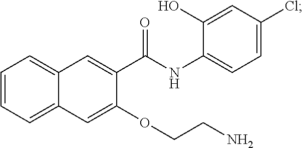 [SMILES: NCCOc1cc2ccccc2cc1C(=O)Nc1ccc(Cl)cc1O]
or a pharmaceutically acceptable salt thereof.

5. A method of treatment in a subject of a cancer or malignant neoplasm associated with or dependent upon CREB-mediated gene transcription, the method comprising administering to the subject in need thereof a therapeutically effective amount of a compound of the formula: 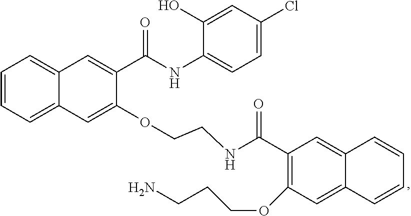 [SMILES: NCCCOc1cc2ccccc2cc1C(=O)NCCOc1cc2ccccc2cc1C(=O)Nc1ccc(Cl)cc1O]
or a pharmaceutically acceptable salt thereof;
and a therapeutically effective amount of a compound of Formula (I): 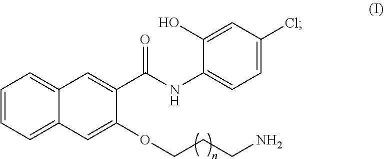
wherein n is an integer selected from the group of 0, 1, 2, 3, and 4, or a pharmaceutically acceptable salt thereof.

6. The method of claim 5, wherein the compound of Formula (I) is the compound: 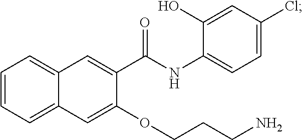 [SMILES: NCCCOc1cc2ccccc2cc1C(=O)Nc1ccc(Cl)cc1O]
or a pharmaceutically acceptable salt thereof.

7. The method of claim 5, wherein the compound of Formula (I) is the compound: 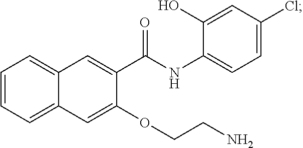 [SMILES: NCCOc1cc2ccccc2cc1C(=O)Nc1ccc(Cl)cc1O]
or a pharmaceutically acceptable salt thereof.

8. The method of claim 5, wherein the cancer or malignant neoplasm associated with or dependent upon CREB-mediated gene transcription is non-small cell lung cancer.

9. The method of claim 5, wherein the cancer or malignant neoplasm associated with or dependent upon CREB-mediated gene transcription is acute myeloid leukemia.

10. A pharmaceutical composition comprising a therapeutically effective amount of a compound of the formula: 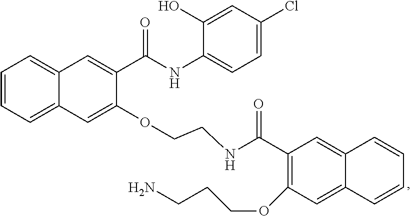 [SMILES: NCCCOc1cc2ccccc2cc1C(=O)NCCOc1cc2ccccc2cc1C(=O)Nc1ccc(Cl)cc1O]
or a pharmaceutically acceptable salt thereof;
and a therapeutically or synergistically effective amount of a compound of Formula (I): 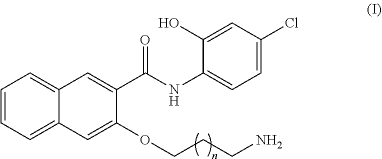
wherein n is an integer selected from the group of 0, 1, 2, 3, and 4, or a pharmaceutically acceptable salt thereof.

11. The pharmaceutical composition of claim 10, wherein the compound of Formula (I) is the compound: 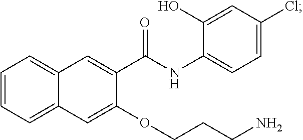 [SMILES: NCCCOc1cc2ccccc2cc1C(=O)Nc1ccc(Cl)cc1O]
or a pharmaceutically acceptable salt thereof.

12. The pharmaceutical composition of claim 10, wherein the compound of Formula (I) is the compound: 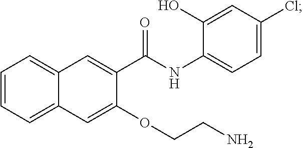 [SMILES: NCCOc1cc2ccccc2cc1C(=O)Nc1ccc(Cl)cc1O]
or a pharmaceutically acceptable salt thereof.
